SEQUENCE LISTING (7 sequences)

SEQ ID NO: 1 - protein, 217 aa, Plasmodium falciparum
MNKLYSLFLFLFIQLSIKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNILNLSIMFILFSVCFFIM

SEQ ID NO: 2 - protein, 185 aa, artificial
  Recombinant Plasmodium falciparum Pfs25H
EAEAYVKVTVDTVCKRGFLIQMSGHLECKCENDLVLVNEETCEEKVLKCDEKTVNKPCGDFSKCIKIDGNPVSYACKCNLGYDMVNNVCIPNECKQVTCGNGKCILDTSNPVKTGVCSCNIGKVPNVQDQNKCSRDGETKCSLKCLKEQETCKAVDGIYKCDCKDGFIIDQESSICTDPHHHHHH

SEQ ID NO: 3 - protein, 219 aa, Plasmodium vivax
MNSYYSLFVFFLVQIALKYSKAAVTVDTICKNGQLVQMSNHFKCMCNEGLVHLSENTCEEKNECKKETLGKACGEFGQCIENPDPAQVNMYKCGCIEGYTLKEDTCVLDVCQYKNCGESGECIVEYLSEIQSAGCSCAIGKVPNPEDEKKCTKTGETACQLKCNTDNEVCKNVEGVYKCQCMEGFTFDKEKNVCLSYSVFNILNYSLFFIILLVLSYVI

SEQ ID NO: 4 - protein, 181 aa, artificial
  Recombinant Plasmodium vivax Pvs25
YSASAVTVDTICKNGQLVQMSNHFKCMCNEGLVHLSENTCEEKNECKKETLGKACGEFGQCIENPDPAQVNMYKCGCIEGYTLKEDTCVLDVCQYKNCGESGECIVEYLSEIQSAGCSCAIGKVPNPEDEKKCTKTGETACQLKCNTDNEVCKNVEGVYKCQCMEGFTFDKEKNVCLGPLC

SEQ ID NO: 5 - protein, 218 aa, Plasmodium falciparum
MNTYFKVLLFLFIQLYITLNKARVTENTICKYGYLIQMSNHYECKCIEGYVLINEDTCGKKVVCDKVENSFKACDEYAYCFDLGNKNNEKQIKCMCRTEYTLTAGVCVPNVCRDKVCGKGKCIVDPANSLTHTCSCNIGTILNQNKLCDIQGDTPCSLKCAENEVCTLEGNYYTCKEDPSSNGGGNTVDQADTSYSVINGVTLTHVLIVCSIFIKLLI

SEQ ID NO: 6 - protein, 448 aa, Plasmodium falciparum
MMLYISAKKAQVAFILYIVLVLRIISGNNDFCKPSSLNSEISGFIGYKCNFSNEGVHNLKPDMRERRSIFCTIHSYFIYDKIRLIIPKKSSSPEFKILPEKCFQKVYTDYENRVETDISELGLIEYEIEENDTNPNYNERTITISPFSPKDIEFFCFCDNTEKVISSIEGRSAMVHVRVLKYPHNILFTNLTNDLFTYLPKTYNESNFVSNVLEVELNDGELFVLACELINKKCFQEGKEKALYKSNKIIYHEKLTIFKAPFYVTSKDVNTECTCKFKNNNYKIVLKPKYEKKVIHGCNFSSNVSSKHTFTDSLDISLVDDSAHISCNVHLSEPKYNHLVGLNCPGDIIPDCFFQVYQPESEELEPSNIVYLDSQINIGDIEYYEDAEGDDKIKLFGIVGSIPKTTSFTCICKKDKKSAYMTVTIDSAYYGFLAKTFIFLIVAILLYI

SEQ ID NO: 7 - protein, 236 aa, Plasmodium vivax
MNTYHSLLFLLAIVLTVKHTFAKVTAETQCKNGYVVQMSNHFECKCNDGFVMANENTCEEKRDCTNPQNVNKNCGDYAVCANTRMNDEERALRCGCILGYTVMNEVCTPNKCNGVLCGKGKCILDPANVNSTMCSCNIGTTLDESKKCGKPGKTECTLKCKANEECKETQNYYKCVAKGSGGEGSGGEGSGGEGSGGEGSGGEGSGGDTGAAYSLMNGSAVISILLVFAFFMMSLV

(19) United States
(12) Patent Application Publication
(10) Pub. No.: US 2009/0047303 A1
(43) Pub. Date: Feb. 19, 2009

(54) Method for improving the immunogenicity of plasmodium antigens

(76) Inventors: Alan Shaw, Doylestown, PA (US); Craig T. Przysiecki, Lansdale, PA (US); Elizabeth M. Flanagan, Minneapolis, MN (US); Roxana Ionescu, Collegeville, PA (US); Li Shi, Eagleville, PA (US); Yimin Wu, Frederick, MD (US); Allan Saul, Rockville, MD (US); Louis Miller, Cabin John, MD (US)

Correspondence Address: MERCK AND CO., INC, P O BOX 2000, RAHWAY, NJ, 07065-0907, US

(21) Appl. No.: 11/919,610

(22) PCT Filed: May 15, 2006
(86) PCT No.: PCT/US2006/018615

§ 371 Date: Oct. 30, 2007

Related U.S. Application Data
(60) Provisional application No. 60/681,546, filed on May 16, 2005.

(51) Int. Cl. A61K 39/00; C07K 14/00; A61P 31/00

(52) U.S. Cl. 424/194.1; 530/350

(57) ABSTRACT

The present invention provides a Malaria antigen-carrier conjugate, which comprises a carrier protein and a plurality of Plasmodium antigen polypeptides. Each of the antigen polypeptides is a wild-type antigen protein of Plasmodium or a derivative of the wild-type antigen protein, and each of the antigen polypeptides may be the same, or different. The plurality of Plasmodium antigen polypeptides are covalently linked to the carrier protein. The present invention further provides a vaccine against malaria, which comprises the conjugate absorbed on an aluminum adjuvant.

### CROSS-REFERENCE TO RELATED APPLICATIONS

The present application claims priority to provisional application U.S. Ser. No. 60/681,546, filed May 16, 2006, hereby incorporated by reference herein.

### BACKGROUND OF THE INVENTION

Malaria is one of most dangerous infectious diseases in tropical and subtropical countries, afflicting about 300 million people. Humans infected with malaria can develop a wide range of symptoms. These vary from asymptomatic infections (no apparent illness in semi-immune adults), to the classic symptoms of malaria (fever, chills, sweating, headaches, and muscle pains), and to severe complications (cerebral malaria and anemia) that can result in death. Malaria kills about one to two million people per year. The majority of the victims are immune-naïve children under the age of five. Pregnant women are particularly susceptible to malaria infection, which results in miscarriage, stillbirth, or low birth-weight infants who are less likely to survive beyond one year. Malaria also slows economic development of developing countries with large population of infected people. (http://www.cdc.gov/malaria/index.htm; Richie and Saul, Nature 415:694-701 (2002); Stowers and Carter, Expert Opin. Biol. Ther. 1:619-628 (2001).

The pathogen of the disease is a protozoan parasite, Plasmodium sp. which is transmitted by Anopheles mosquitoes. Four species of malaria parasites can infect humans under natural conditions: Plasmodium falciparum, P. vivax, P. ovale, and P. malariae. While the first two species cause the most infections worldwide, P. falciparum is the agent of severe, potentially fatal malaria. (ibid.)

Plasmodium has a complex life-cycle, encompassing asexual reproduction in human hosts (pre-erythrocytic and erythrocytic stages) and sexual reproduction in mosquito hosts (referred to as the sexual, or, more broadly, as the mosquito stage). Infection in humans follows the inoculation of sporozoites by an infected Anopheles mosquito. After a brief period in the blood, the sporozoites invade hepatocytes where they undergo a process of development and asexual multiplication known as schizogony. At the end of this pre-erythrocytic stage, the infected hepatocyte ruptures and merozoites are released into the blood circulation. The erythrocytic stage begins when the merozoites invade erythrocytes where they feed on host nutrients, develop from a ring form, to a trophozoite form, and undergo multiple nuclear divisions to form a schizont. At the end of schizogony, the erythrocyte ruptures and the released merozoites then invade other erythrocytes. The asexual erythrocytic stage is the cause of the characteristic malarial fever and death. During the erythrocytic stage, some merozoites develop into sexually differentiated gametocytes. The male and female gametocytes, after being ingested by Anopheles mosquitoes through a subsequent bite, develop and fertilize to form a zygote in the mosquito midgut. The zygote develops into an elongated form, the ookinete, which later passes through midgut epithelial cells and forms an oocyst. The oocyst undergoes repeated karyo- and cytokinesis, producing thousands of sporozoites that, upon maturation, migrate to the salivary gland of the mosquito, and transmit to the next human host when the mosquito feeds again. (Richie and Saul, Nature 415:694-701 (2002); Stowers and Carter, Expert Opin. Biol. Ther. 1:619-628 (2001); Kaslow, et al., Trends in Biotechnology, 10:388-391 (1992)) Moreover, P. vivax and P. ovale have dormant liver stage forms (“hypnozoites”) which can reactivate (“relapse”) and cause malaria several months or years after the infecting mosquito bite.

Mounting drug-resistance of the malaria parasite makes chemotherapy increasingly difficult. Quinine, chloroquine, mefloquine, atovaquone and proguanil have been widely used in the treatment of malaria. However, the malaria parasite has evolved into resistant strains against these antimalarials. (Stowers and Carter, Expert Opin. Biol. Ther. 1:619-628 (2001); Donovan, HHMI Bulletin, March 2003, 14-19). Consequently, artemisinin-based derivatives have become front-line drug for malaria treatment. Unfortunately, because of wide and indiscriminate use of artemisinin-based derivatives in the endemic countries, the World Health Organization (WHO) anticipates to see malaria forms resistant to artemisinin soon.

In contrast to drug treatment, vaccination is an approach for the prophylaxis and eradication of malaria. In the poorest countries of the world, the development of a vaccine against malaria might be one of the most cost-effective interventions to reduce the enormous burden of the disease. (ibid, Richie and Saul, Nature 415:694-701 (2002); UNDP/World Bank/WHO, TDR/PBM/MAL/VC/2000.1) However, no malaria vaccine is yet available. Different antigens are expressed in the different stages of the malaria parasite, and are thus candidates for malaria vaccines. Problematically, the immunogenicity in humans of these antigens is low. (ibid.)

Hence, there is a need to improve the immunogenicity of Plasmodium antigens to make effective vaccines against malaria.

The references cited herein are not admitted to be prior art to the claimed invention.

### SUMMARY OF THE INVENTION

The present invention provides a malaria antigen-carrier conjugate, which comprises a carrier protein and a plurality of Plasmodium antigen polypeptides. Each of the antigen polypeptides is a wild-type antigen protein expressed in the sexual or mosquito stage of Plasmodium or a derivative of the wild-type antigen protein. Each of the antigen polypeptides may be the same, or may be different. The plurality of Plasmodium antigen polypeptides are covalently linked to the carrier protein. The plurality of Plasmodium antigen polypeptides are preferably covalently linked to the carrier protein via a thioether linker. The Plasmodium species can be P. falciparum, P. vivax, P. ovale, or P. malariae.

The plurality of Plasmodium antigen polypeptides can comprise a derivative from an antigen selected from the group consisting of Pfs25, Pfs28, and Pfs48/45 of P. falciparum. According to an embodiment of the present invention, the plurality of Plasmodium antigen polypeptides comprise a Pfs25H protein produced in Pichia pastoris or Saccharomyces cerevisiae. According to a preferred embodiment of the present invention, the plurality of Plasmodium antigen polypeptides consist essentially of a Pfs25H protein produced in P. pastoris.

Alternatively, the plurality of Plasmodium antigen polypeptides can comprise a derivative from Pvs25 or Pvs28 of P. vivax. According to an embodiment of the present invention, the plurality of Plasmodium antigen polypeptides comprises a Pvs25H protein produced in S. cerevisiae.

According to a preferred embodiment of the present invention, the plurality of Plasmodium antigen polypeptides may comprise derivatives of Pfs25 and Pvs25. According to a more preferred embodiment of the present invention, the plurality of Plasmodium antigen polypeptides consists essentially of derivatives of Pfs25 and Pvs25.

The carrier protein can be selected from the group consisting of OMPC (Outer Membrane Protein Complex of Neisseria meningitidis), BSA (bovine serum albumin), OVA (ovalbumin), THY (bovine thyroglobulin), KLH (keyhole limpet hemocyanin), TT (tetanus toxoid protein), HbSAg (surface antigen protein) and HBcAg (core antigen protein) of Hepatitis B virus, rotavirus capsid proteins, the L1 protein of the human papilloma virus, and VLP (virus-like particle) type 6, 11 and 16. The carrier protein is preferably OMPC, and more preferably improved OMPC (iOMPC).

The present invention also provides a malaria antigen-carrier conjugate, which is produced by a method comprising conjugating to a carrier protein a plurality of Plasmodium antigen polypeptides. Each of the antigen polypeptides is a wild-type antigen protein expressed in the sexual or mosquito stage of Plasmodium or a derivative of the wild-type antigen protein. Each of the antigen polypeptides may be the same, or different. The plurality of Plasmodium antigen polypeptides preferably comprise Pfs25H. The carrier protein is preferably OMPC.

The conjugating can be achieved through a scheme selected from the group consisting of maleimide/thiol coupling, bromoacetamide/thiol coupling, and histidine-selective cross-linking.

The conjugating can be achieved using a cross-linker selected from the group consisting of one of more of sulfosuccinimidyl 4-(N-maleimidomethyl)cyclohexane-1-carboxylate (sSMCC), N-maleimidobenzoyl-N-hydroxysuccinimide ester (MBS), glutaraldehyde, 1-ethyl-3-(3-dimethylaminopropyl)carbodiimide (EDCI), Bis-diazobenzidine (BDB), N-acetyl homocysteine thiolactone (NAHT), and N-[ε-Maleimidocaproyloxy]sulfosuccinimide ester (sEMCS). The cross-linker is preferably N-[ε-Maleimidocaproyloxy]sulfosuccinimide ester (sEMCS).

The present invention further provides a vaccine against malaria comprising the malaria antigen-carrier conjugate, an adjuvant and a physiologically acceptable carrier. The conjugate is preferably a Pfs25H-OMPC conjugate. The conjugate is preferably absorbed onto Merck aluminum adjuvant. The vaccine can further comprise a Pvs25-OMPC conjugate. The conjugate used in the vaccine can comprise a plurality of Plasmodium antigen polypeptides consisting essentially of derivatives of Pfs25 and Pvs25, an adjuvant, and a physiologically acceptable carrier.

The present invention also provides a method for blocking malaria transmission, which comprises administering the vaccine to a subject. The subject is preferably human.

Other features and advantages of the present invention are apparent from the additional descriptions provided herein including the different examples. The provided examples illustrate different components and methodology useful in practicing the present invention. The examples do not limit the claimed invention. Based on the present disclosure the skilled artisan can identify and employ other components and methodology useful for practicing the present invention.

### BRIEF DESCRIPTION OF THE DRAWINGS

FIG. 1: Examples of antigens expressed in P. falciparum, and P. vivax malaria parasites

FIG. 1A: The sequences of wild-type Pfs25, CAA30646 (SEQ ID NO:1), and a recombinant Pfs25, Pfs25H (SEQ ID NO:2). The Pfs25H amino acids in lower case indicates its N-terminal secretory α-factor signal peptide, C-terminal hexahistidine tag, and point mutations of asparagines 112, 165, and 187 to glutamines, respectively.

FIG. 1B: The sequence of wild-type Pvs25, AAC99769 (SEQ ID NO:3), and a recombinant Pvs25, Pvs25H (SEQ ID NO:4);

FIG. 1C: The sequence of Pfs28 (SEQ ID NO:5);

FIG. 1D: The sequence of Pfs48/45 (SEQ ID NO:6);

FIG. 1E: The sequence of Pvs28 (SEQ ID NO:7).

FIG. 2: Conjugation reaction of maleimide activated Pfs25H with thiolated OMPC.

FIG. 3: Unique acid hydrolysis of amino acids of antigens and conjugates

FIG. 3A: Unique acid hydrolysis of amino acids of maleimide-activated antigen,

FIG. 3B: Unique acid hydrolysis of amino acids of N-acetylcysteine quenched-maleimide activated antigen, and

FIG. 3C: Unique acid hydrolysis amino acids of conjugate between maleimide activated antigen and N-acetylhomocysteine activated carrier protein.

FIG. 4: Induction of specific antibody responses in mice by formulated Pfs25 and Pfs25H-OMPC conjugate vaccines. The histograph bars indicate mean antibody levels in these animal groups, with solid dots indicating the antibody level of each animal within each group. The antibody levels are expressed as ELISA units, the reciprocal of the serum dilution which gives an O.D.=1 in a standard ELISA assay tested against a reference antiserum on each plate.

FIG. 5: Pfs25H-OMPC dose and response in mice, rabbits, and monkeys. The histograph indicates mean antibody levels in each animal group, with solid dots indicating the antibody level of each animal in the group. The antibody levels are expressed as ELISA units, the reciprocal of the serum dilution which gives an O.D.=1 in a standard ELISA assay tested against a reference antiserum on each plate.

FIGS. 6A, 6B, and 6C: Antibody responses time course in mice, rabbits, and rhesus monkeys, respectively. Two groups of mice were given one (open circle) or two (open square) immunizations of 0.25 μg of conjugated Pfs25H. The second immunization was given 4 weeks after the first immunization. Three groups of rabbits were given 2 immunizations of 1, 5, or 20 μg of conjugated Pfs25H on study days 0 and 28. For the rhesus study, the animals were given 2 immunizations of 4 or 20 μg conjugated Pfs25H on study days 0 and 70. Sera collected at the indicated time points were analyzed for anti-Pfs25H levels.

### DETAILED DESCRIPTION OF THE INVENTION

In the present invention, the immunogenicity of a Plasmodium antigen polypeptide is increased, preferably by conjugating the polypeptide to a carrier protein, which inherently has a high immunogenicity. The immunogenicity—may be further increased by absorbing the conjugate onto an adjuvant, such as an aluminum adjuvant.

### 1. The Plasmodium Antigen Polypeptide

The antigen can be from any species of the malaria parasite Plasmodium, including P. falciparum, P. vivax, P. ovale and P. malariae. The Plasmodium is preferably P. falciparum, and/or P. vivax, more preferably P. falciparum. The genome of Plasmodium falciparum has been completely sequenced. (Gardner et al., Nature, 419:498-511 (2002)).

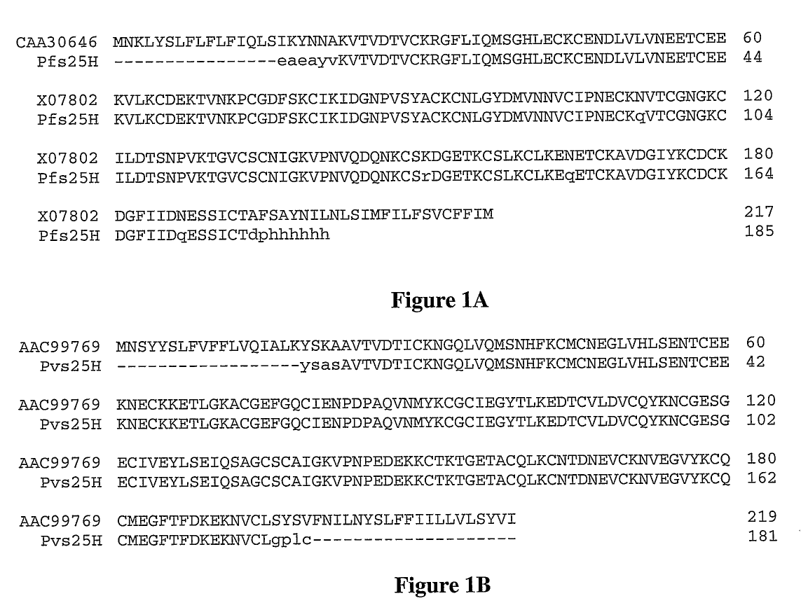

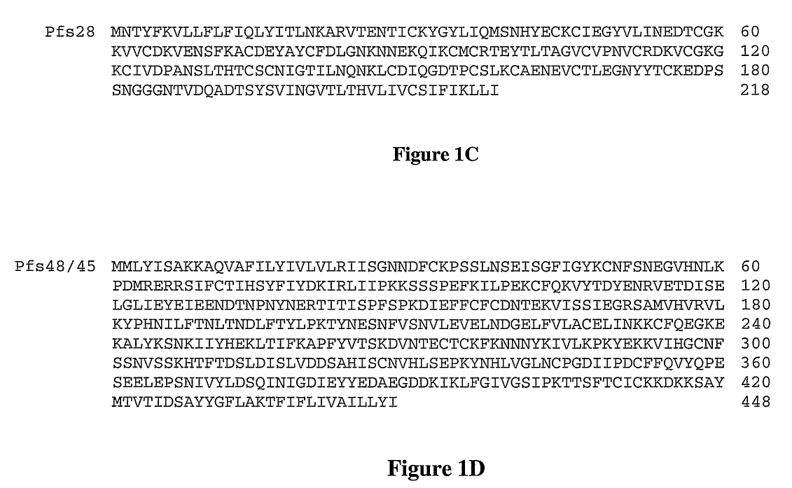

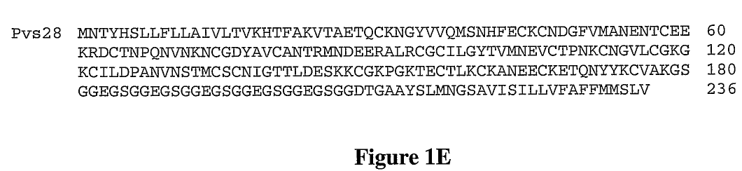

The antigen is preferably expressed in the sexual or mosquito stage of Plasmodium in the gut of mosquito (ibid.; Kaslow et al, Nature 333:74-76 (1988); Duffy and Kaslow, Infect. Immun. 65:1109-1113 (1997); Tsuboi, et al, Mol Med. 4:772-82. (1998); Hisaeda, et al, Infect. Immun., 68:6618-6623 (2000)). More preferably, the antigen is selected from the group consisting of Pfs25, Pfs28, and Pfs48/45 of P. falciparum, and Pvs25 and Pvs28 of P. vivax. (FIG. 1A-1E). Most preferably, the antigen is Pfs25 of P. falciparum. Alternatively, the antigen is Pvs25 of P. vivax.

Pfs25 is a P. falciparum antigen expressed on the surface of the malaria parasite. Pfs25 consists of four epidermal growth factor (EGF)-like domains located between a secretory signal sequence at the N-terminus, and a short C-terminal hydrophobic domain, which seems involved in the transfer of the EGF-like domains to a glycosyl-phosphatidylinositol (GPI) lipid anchor. There are 22 cysteine residues present as disulfide bonds in the four EGF-like domains of Pfs25. (Kaslow, et al., Trends in Biotechnology, 10:388-391 (1992); Kaslow, et al., Nature, 333:74-76 (1988)). The disulfide bonds between the cysteine residues are essential for maintaining the structural integrity of the antigen. In an ex vivo experiment, antibodies to the antigen can completely block transmission of P. falciparum. (ibid, Vermeulen, et al., J. Exp. Med. 162:1460-1476 (1985)) The counterpart of Pfs25 in P. vivax is Pvs25.

Because Pfs25 is expressed only in P. falciparum at the sexual or, more precisely, the mosquito stage in the mosquito, the antigen might never be under host immune selection pressure, and have not evolved sequence diversity to evade the human immune system. Indeed, Pfs25 is highly conserved among strains from a wide range of endemic regions. Thus, Pfs25 does not have the antigen polymorphism and geographical diversity problems that exist in other Plasmodium antigens expressed in the asexual or blood stage in the human. (Kaslow, et al., Trends in Biotechnology, 10:388-391 (1992); Kaslow, Immunol. Lett. 25:83-86 (1990))

As used herein, a “Plasmodium antigen polypeptide” can be a wild-type Plasmodium antigen protein expressed in the sexual or mosquito stage, or a derivative of the wild-type antigen protein. As used herein, “a derivative from an antigen expressed in Plasmodium” is a recombinant polypeptide, which comprises at least 10 amino acids with a sequence identity of at least 90% identity to a reference sequence.

The reference sequence can be the sequence of a Plasmodium antigen expressed in the sexual or mosquito stage (e.g., SEQ ID NO:1), or a portion of the sequence with at least 10 amino acids. Sequence identity to reference sequence is determined by aligning the amino acid sequence of the derivative with the reference sequence, and determining the number of identical amino acids in the corresponding positions of the two sequences. This number is divided by the total number of amino acids in the reference sequence, and then multiplied by 100 and rounded to the nearest whole number.

In a different embodiment, the derivative comprises a sequence of at least 20, 30, 50, 80, 100, 150, or 200 amino acids, with a sequence identity of at least 90% or 95%, or 100%. The derivative may consist essentially of a sequence of at least 20, 30, 50, 80, 100, 150, or 200 amino acids, with a sequence identity of at least 90% or 95%, or 100%. The derivative may consist of a sequence of at least 20, 30, 50, 80, 100, 150, or 200 amino acids, with a sequence identity of at least 90% or 95%, or 100%. Each alteration to a reference sequence is independently an addition, deletion, or substitution. The number and position of permissible alterations will vary depending on antigen. For example, for Pfs25, it is important to maintain its secondary structure. Examples of permissible alterations for Pfs25 are provided by Pfs25H (FIG. 1A).

Pfs25H, a recombinant form of the Pfs25 protein, is expressed in both S. cerevisiae, and P. pastoris. (Kaslow, et al., Trends in Biotechnology, 10:388-391 (1992); Zou, et al., Vaccine 21:1650-1657 (2003)). In Pfs25H, the original Pfs25 signal peptide was replaced with a secretory α-factor signal peptide, and the original Pfs25 C-terminal hydrophobic sequence was replaced with a C-terminal hexahistidine tag to facilitate purification. Moreover, the three potential N-linked glycosylation sites were removed with asparagines 112, 165, and 187 mutated to glutamines. In P. pastoris, the resulting recombinant Pfs25H protein has a sequence shown in FIG. 1A. The Pfs25H expressed in P. pastoris can elicit antibodies in mice, which are capable of blocking transmission in ex vivo tests (Zou, et al., Vaccine 21:1650-1657 (2003)).

Recombinant Pfs25 has been also expressed in recombinant vaccinia virus system. (Kaslow, et al., Trends in Biotechnology, 10:388-391 (1992))

### 2. The Carrier Proteins

The carrier proteins used in the present invention can be any protein carrier used in the art. Examples of well-known carrier proteins include OMPCOuter Membrane Protein Complex of Neisseria meningitidis, BSA (bovine serum albumin), OVA (ovalbumin), THY (bovine thyroglobulin), KLH (keyhole limpet hemocyanin), TT (tetanus toxoid protein), Hepatitis B virus proteins including the surface antigen protein (HBsAg) and the core antigen protein (HBcAg), rotavirus capsid proteins and the L1 protein of the human papilloma virus, VLP (virus-like particle) type 6, 11 or 16, etc.

Preferably, the carrier protein is OMPC of Neisseria meningitidis, which is a very effective immunogenic carrier derived from N. meningitidis serogroup B strain B11. OMPC is approved by regulatory agencies worldwide as a vaccine component for polysaccharide antigens. OMPC is used in the Merck PedvaxHiB® process. (Marburg, et. al. J. Am. Chem. Soc. 108:5282-5287 (1986)).

Various methods of purifying OMPC from the gram-negative bacteria have been devised (Frasch et al., J. Exp. Med. 140, 87 (1974); Frasch et al., J. Exp. Med. 147, 629 (1978); Zollinger et al., U.S. Pat. No. 4,707,543 (1987); Helting et al., Acta Path. Microbiol. Scand. Sect. C. 89, 69 (1981); Helting et al., U.S. Pat. No. 4,271,147). N. meningitidis serogroup B improved Outer Membrane Protein Complex (iOMPC) can be obtained using techniques well known in the art such as those described by Fu, U.S. Pat. No. 5,494,808.

### 3. The Conjugation Methods

The peptides and the carrier proteins of the present invention can be conjugated using conjugation chemistry well known in the art. According to an embodiment of the present invention, the conjugation can be achieved using sulfosuccinimidyl 4-(N-maleimidomethyl)cyclohexane-1-carboxylate (sSMCC), N-maleimidobenzoyl-N-hydroxysuccinimide ester (MBS), glutaraldehyde, 1-ethyl-3-(3-dimethylaminopropyl)carbodiimide (EDCI), Bis-diazobenzidine (BDB), and/or N-[ε-maleimidocaproyloxy]sulfosuccinimide ester (sEMCS). Other conjugation methods and reagents can be found in U.S. Pat. No. 5,606,030. Different conjugation methods and reagents usually lead to different linkages between the peptides and the carrier protein.

The conjugation method should be selected based on the characteristics of the carrier protein and the Plasmodium antigen polypeptide to be conjugated. The linkage should not interfere with the desired epitope in the polypeptide. Within the conjugated antigen polypeptide, the cross-linked residue is preferably separated from the desired epitope sequence with a distance of at least one amino acid as a spacer.

### 3.1. Maleimide/Thiol Coupling Scheme

Maleimide/thiol coupling scheme can be used to conjugate the Plasmodium antigen polypeptide and the carrier protein. The conjugation scheme can be achieved using sulfosuccinimidyl 4-(N-maleimidomethyl)cyclohexane-1-carboxylate (sSMCC), N-maleimidobenzoyl-N-hydroxysuccinimide ester (MBS), or N-[ε-maleimidocaproyloxy]sulfosuccinimide ester (sEMCS).

3.1.1. Coupling Method Using sSMCC

The method using sSMCC is widely used and highly specific (see, e.g., Meyer et al. 2002, J. of Virol. 76, 2150-2158 (2002)). sSMCC can be used to cross-link the SH-group (e.g. cysteine residue) of a peptide antigen to the amino group (e.g., epsilon-amino of lysine residue) on the carrier protein. In the conjugation reaction using sSMCC, the carrier protein is first activated with sSMCC reagent by acylating the lysine residues and N-terminal α-amino groups of the carrier protein. After separation of the activated carrier protein from the excess reagent and the by-product, the cysteine-containing peptide is added and the covalent link takes place by Michael addition of the SH-group to the double bond of maleimide functionality of the activated carrier protein. The method using MBS or sEMCS conjugates the peptide and the carrier protein through a similar mechanism.

The maleimide/thiol coupling scheme is highly specific for SH-groups, especially at low reaction pH values. If an antigen polypeptide does not have a cysteine residue, a cysteine residue could be added to the polypeptide, preferably at the N-terminus or C-terminus. If the desired epitope in the polypeptide contains a cysteine, the polypeptide should not be reacted with a maleimide-activated carrier protein. If the polypeptide contains more than one cysteine residue, the polypeptide should not be reacted with a maleimide-activated carrier protein, unless the excess cysteine residues can be replaced or modified.

Recombinant Pfs25H does not contain any detectable free thiol. Intramolecular disulfide bonds formed among Pfs25 cysteine residues might be essential for the conformation of desired epitopes on the Plasmodium antigen polypeptide. Addition of an extra thiol group to Pfs25 for coupling purposes might potentially disrupt critical disulfides.

To solve this problem, the maleimide/thiol coupling scheme can be modified by maleimide-activating the Plasmodium antigen polypeptide instead. Potential thiol-disulfide exchange with a free thiol on a carrier protein should be minimized by lowering the pH of the conjugation reaction. Maleimide/thiol coupling chemistry was preferred because of its better reaction efficiency at pH˜6.

3.1.2. Coupling Method Using sEMCS

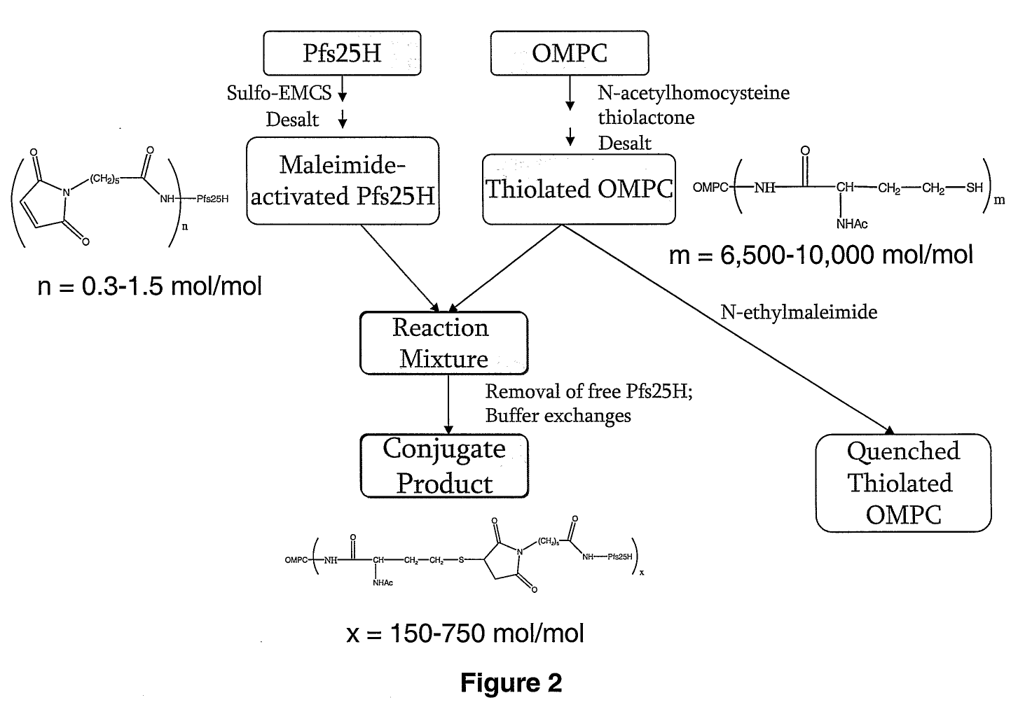

Another heterobifunctional cross-linker, N-[ε-maleimidocaproyloxy]sulfosuccinimide ester (sEMCS), can be used to maleimide-activate the Plasmodium antigen polypeptide, as long as the antigen polypeptide contains usable lysine residues or N-terminal α-amino groups for conjugation. sEMCS has a flexible linker structure with suggested lower immunogenicity potential (Peeters et al. J. of Immunological Methods 120, 133-143 (1989)). In the conjugation reaction, the antigen polypeptide is activated by the sEMCS reagent acylating amines of the antigen polypeptide. The level of sEMCS acylation is low to minimize potential disruption of epitopes. After separation of the activated antigen polypeptide from the excess reagent and the by-product, the maleimide-activated antigen is added to a free thiol cysteine-containing carrier protein or a thiolated carrier protein. The maleimide-activated antigen polypeptide is coupled to thiolated carrier protein by formation of thioether bond between the maleimide and the N-acetylhomocysteine thiols of thiolated carrier protein or free thiol cysteines of a carrier protein. (FIG. 2)

N-acetyl homocysteine thiolactone (NAHT) can be used to introduce a thiol functionality onto the carrier protein, to allow conjugation with maleimidated or bromo-acetylated-peptides (Tolman et al. Int. J. Peptide Protein Res. 41:455-466 (1993); Conley et al. Vaccine 12:445-451 (1994)). (FIG. 2)

### 3.2. Bromoacetamide/Thiol Coupling Scheme

Similarly, bromoacetamide-activated antigen polypeptide can also be prepared and reacted with thiolated carrier protein. (Kolodny and Robey, Anal. Biochem 187, 136-140. (1990)) However, the higher pH needed for efficient reaction with bromoacetamide-activated antigen polypeptide may be less preferred for an antigen like Pfs25; since there is greater potential for thiol-disulfide interchange at higher pH values. The reactive groups can be switched where the antigen possesses the thiol group and the carrier protein is bromoacetamide activated (Bernatowicz and Matsueda, Anal. Biochem. 155, 95-102 (1986)). The same potential issue with thiol disulfide exchange at a higher reaction pH would apply.

### 3.3. Other Coupling Scheme

Histidine-selective cross-linkers and/or conditions could also be used. The cross-linker takes advantage of usable histidine residue in the sequence of the antigen polypeptide, or a His-tag, which is widely adopted in the terminus of recombinant protein to facilitate purification. At lower reaction pH (5 to 6) in absence of free thiols, alkyl halides can selectively react with either nitrogen of the imidazole group of certain histidine residues (Yamada, et al. J. Biochem. 95, 503-510 (1984)).

### 4. Vaccines Against Malaria

The present invention can be used to produce conjugates of Plasmodium antigen polypeptide, such as Pfs25H-OMPC conjugates. The conjugates can then be used in vaccination against malaria.

### 4.1. Malaria Infection-Blocking Vaccine and Malaria Transmission-Blocking Vaccine

As described above, different antigens are expressed in the pre-erythrocytic, erythrocytic, and mosquito stages of the malaria parasite life-cycle. Malaria vaccine candidates can be selected from the antigens expressed in any of the three stages. Most current efforts focus on the antigens expressed in the stages of the parasite's life cycle that reside in the human host (i.e., pre-erythrocytic and erythrocytic stages). An immune response against such antigens could completely preclude initiation of the infection process, or would most likely control disease through reduction of parasite load within an individual. (Richie and Saul, Nature 415:694-701 (2002); Nussenzweig and Zavala, NEJM 336:128-130 (1997))

In contrast, the antigens expressed at the sexual or mosquito stage in the mosquito can be used to develop malaria transmission-blocking vaccines. In this strategy, antibodies against said antigens are produced in human bodies through vaccination, but cannot block the malaria infection of human directly. The antibodies and complement in vaccinated individuals would be taken up along with the blood meal by the mosquito. Functional antibodies would prevent parasite development within the mosquito, and thereby block the transmission of the parasite to another human. (Kaslow, et al., Trends in Biotechnology, 10:388-391 (1992); Stowers and Carter, Expert Opin. Biol. Ther. 1:619-628 (2001); UNDP/World Bank/WHO, TDR/PBM/MAL/VC/2000.1)

The conjugates of the present invention may overcome a major hurdle in the development of malaria vaccine by increasing the limited immunogenicity in humans of malaria vaccine candidates, i.e., the antigens expressed in the sexual or mosquito stage of the malaria parasite. Hence, the conjugates of the present invention can be utilized to make malaria transmission-blocking vaccines.

The vaccine candidates can be used singly, or in combinations that can be classified as multistage vaccine cocktails. A vaccine cocktail comprising both human-stage antigens and the conjugates of the present invention will not only protect the vaccinated individuals from malaria infection, but also prevent malaria transmission in the local community. Further, a vaccine cocktail comprising conjugates of antigen polypeptides derived from different Plasmodium species will be capable of blocking the infection and/or transmission of more than one Plasmodium species. For example, a vaccine cocktail comprising Pfs25 conjugate and Pvs25 conjugate will be capable of blocking the transmission of both P. falciparum and P. vivax, the two species that cause the most infections worldwide.

In addition to vaccines, the conjugates of the present invention can be used in other ways, including generating antibodies for passive immunization and as reagents for immunoassays.

### 4.2. Vaccine Formulations

The conjugates of the present invention can be used as vaccines to immunize mammals including humans against infection and/or transmission of malaria parasite, or to treat humans post-infection, or to boost a pathogen-neutralizing immune response in a human afflicted with infection of malaria parasite.

The vaccine of the present invention can be formulated according to methods known and used in the art. Guidelines for pharmaceutical administration in general are provided in, for example, Modern Vaccinology, Ed. Kurstak, Plenum Med. Co. 1994; Remington's Pharmaceutical Sciences 18th Edition, Ed. Gennaro, Mack Publishing, 1990; and Modern Pharmaceutics 2nd Edition, Eds. Banker and Rhodes, Marcel Dekker, Inc., 1990.

Conjugates of the present invention can be prepared as various salts. Pharmaceutically acceptable salts (in the form of water- or oil-soluble or dispersible products) include conventional non-toxic salts or the quaternary ammonium salts that are formed, e.g., from inorganic or organic acids or bases. Examples of such salts include acid addition salts such as acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate; and base salts such as ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as histidine, arginine and lysine.

It is preferred that the adjuvant is chosen as appropriate for use with the particular carrier protein used in the conjugate as well as the ionic composition of the final formulation. Consideration should also be given to whether the conjugate alone will be formulated into a vaccine or whether the conjugate will be formulated into a combination vaccine. In the latter instance one should consider the buffers, adjuvants and other formulation components that will be present in the final combination vaccine.

It is preferred that the vaccine be formulated with an aluminum adjuvant. Aluminum based adjuvants are commonly used in the art and include aluminum phosphate, aluminum hydroxide, aluminum hydroxy-phosphate, and amorphous aluminum hydroxyphosphate sulfate. Trade names of aluminum adjuvants in common use include ADJUPHOS®, ALHYDROGEL®, (both from Superfos Biosector a/s, DK-2950 Vedbaek, Denmark), and Merck aluminum adjuvant (MAA, amorphous aluminum hydroxyphosphate sulfate, Merck & Co. West Point, Pa., USA) (Ruiz, et al, J Immune Based Ther Vaccines 3:2 (2005); Koutsky, et al, N Engl J Med, 347:1645-1651 (2002)) The conjugate can be bound to or co-precipitated with the adjuvant as desired and as appropriate for the particular adjuvant used.

For example, after conjugate purification, the Pfs25H-OMPC conjugate is adsorbed onto MAA. In mice (MVDB/NIAID), the conjugate provided a higher functional antibody response to Pfs25H compared to unadjuvanted Pfs25H, free Pfs25H adsorbed to MAA, or Pfs25H formulated with non-aluminum adjuvants. Adsorption of free Pfs25H onto MAA gave a more stable binding than apparently observed with other types of aluminum adjuvants (MVDB/NIAID).

Non-aluminum adjuvants can also be used. Non-aluminum adjuvants include QS21, Lipid-A, Iscomatrix®, and derivatives or variants thereof, Freund's complete or incomplete adjuvant, neutral liposomes, liposomes containing vaccine and cytokines or chemokines.

In other preferred embodiments, the vaccine is formulated with both an aluminum adjuvant and QS21 or Iscomatrix®. It is preferable, in certain embodiments, to formulate the conjugates with immunogens from Haemophilus influenza, hepatitis viruses A, B, or C, human papilloma virus, measles, mumps, rubella, varicella, rotavirus, Streptococcus pneumonia and Staphylococus aureus. Additionally, the vaccine of the present invention can be combined with other antigenic components of the same pathogen. In this manner a combination vaccine can be made. Combination vaccines have the advantages of increased patient comfort and lower costs of administration due to the fewer inoculations required.

When formulating combination vaccines one should be mindful of the various buffers and adjuvants used with the other immunogens. Some buffers may be appropriate for some immunogen-adjuvant pairs and not appropriate for others. In particular, one should assess the effects of phosphate levels on the various immunogen-adjuvant pairs to assure compatibility in the final formulation.

### 4.3. Vaccine Development

The formulated conjugates, such as MAA adsorbed Pfs25H-OMPC, were used for animal immunogenicity studies including mice, rabbits, and Rhesus monkeys. As shown in the examples, the immunogenicity of the Pfs25H was improved about 50 times by the conjugation to OMPC.

Initial maximal conjugate dose and route of administration in mice was determined by previous work with free Pfs25H, and Pfs25H formulated with Montanide® ISA720 (MVDB/NIH, unpublished). Based on initial conjugate immunogenicity results dose ranging in mice was performed as well as examining other routes of administration. Doses for other animals were based on previous experience with other OMPC conjugate vaccines and/or established protocols (MVDB/NIH, unpublished). Levels of anti-antigen antibodies in vaccinated animal sera can be determined by various assays including ELISA or multiplexing spectroscopic based detection methods (e.g. Luminex). Functional properties of the vaccine induced serum antibodies can be tested in an ex vivo mosquito feeding assay. The assay readout is reduction in the number of oocysts in the mid-gut of the mosquito. (Collins et al, Mosquito News 24: 28-31 (1964); Barr, et al., J Exp Med, 174:1203-1208 (1991); Kaslow, et al., Mem Inst Oswaldo Cruz, 87:175-177 (1992))

In humans, the vaccine would be part of an effort to control malaria. Anti-Pfs25H antibodies in vaccinated individuals would be taken up along with the blood meal by the mosquito. Functional antibodies would prevent parasite oocyst formation within the mosquito blocking the transmission of the parasite to another human.

### 4.4. Vaccine Administration

The vaccines of the present invention can be administered to a patient by different routes such as oral, subcutaneous, or intramuscular. A preferred route is intramuscular. Suitable dosing regimens are preferably determined taking into account factors well known in the art including age, weight, sex and medical condition of the subject; the route of administration; the desired effect; and the particular conjugate employed (e.g., the protein, the protein loading on the carrier protein, etc.).

The conjugates of this invention, when used as a vaccine, are to be administered in immunologically effective amounts. An immunologically effective dose is one that stimulates the immune system of the patient to establish a level of immunological response sufficient to reduce parasite density and disease burden caused by infection with the pathogen, and/or sufficient to block the transmission of the pathogen in human. The vaccine can be used in multi-dose vaccination formats. It is expected that a dose would consist of the range of 1 μg to 1.0 mg total protein. In an embodiment of the present invention the range is 0.1 mg to 1.0 mg. However, one may prefer to adjust dosage based on the amount of peptide delivered. In either case these ranges are guidelines. More precise dosages should be determined by assessing the immunogenicity of the conjugate produced so that an immunologically effective dose is delivered. The conjugate is preferably formulated with an adjuvant.

The timing of doses depends upon factors well known in the art. After the initial administration one or more booster doses may subsequently be administered to maintain antibody titers. An example of a dosing regime would be day 1, 1 month, a third dose at either 4, 6 or 12 months, and additional booster doses at distant times as needed.

A patient or subject, as used herein, is a mammal and preferably a human. A patient can be of any age at which the patient is able to respond to inoculation with the present vaccine by generating an immune response. The immune response so generated can be completely or partially protective against debilitating symptoms caused by infection with a pathogen such as Plasmodium falciparum, and/or can block transmission of the pathogen by Anopheles mosquitoes.

### EXAMPLES

Examples are provided below to further illustrate different features of the present invention. The examples also illustrate useful methodology for practicing the invention. These examples do not limit the claimed invention.

N. meningitidis serogroup B improved Outer Membrane Protein Complex (iOMPC) was obtained using techniques described by Fu, U.S. Pat. No. 5,494,808, herein incorporated by reference in its entirety.

Recombinant Pfs25H is expressed as a secreted product from yeast Pichia pastoris and is provided as a sterile purified product in saline by MVDB/NIAID. (Zou, et al., Vaccine 21:1650-1657 (2003)).

Statistical analyses of the immunization data were performed using software package UNISTAT (P-STAT Inc., Hopewell, N.J.). Probability values less than 0.05 are considered as significant. Mann-Whitney U test was used for comparing antibody titers of two groups. Spearman rank correlation was used for testing correlation between vaccine dose and the antibody levels, the antibody titer and the transmission blocking activity expressed as percentage of inhibition on parasite development in mosquitoes. Curve fitting analyses were performed using Sigma Plot (SPSS Inc., Chicago, Ill.).

### Example 1

### Thiolation of iOMPC

iOMPC was thiolated with N-acetyl homocysteine thiolactone (NAHT). For a general description of OMPC thiolation see Marburg et al. J. Am. Chem. Soc. 108:5282-5287 (1986). The thiolated iOMPC had 1.6 mM thiol equivalents, and had a concentration of 9.3 mg protein/mL. Thiolated iOMPC is made to be 25 mM MES, pH 6.1 buffer for the conjugation reaction. The process was carried out aseptically using sterile iOMPC.

### Example 2

### Maleimide-Activation of Pfs25H

Pfs25H was activated with sulfo-EMCS at pH 7.3 with a 2.5 fold mol excess reagent. Pfs25H has twenty-one lysine residues. The ε-amino group of lysine and the α-amino group of the N-terminal would be the likely sites for reaction of the activated ester of heterobifunctional sEMCS. Maleimide activation level was kept low so that activation did not modify too many amine residues in Pfs25H, which could destroy epitopes, and to lower the possibility of thiolated iOMPC cross-linking reactions through the multivalent activated Pfs25H. Ideally, a single maleimide activation/Pfs25H would be obtained but higher level would also be useful. Since multiple amines exist in Pfs25, the maleimide activation is heterogeneous with respect to number and specific amines activated. Activation with a 2.5 fold excess of sulfo-EMCS at lower pH values (pH 6.1 and 5.5) yields lower levels of activation as expected. However, it may also lead to a more selective activation at the N-terminal α-amino group or ε-amino group of lysine with abnormally low pKa values. Activation at lower pH values and higher reagent ratio seems to yield a more selective maleimide activated Pfs25.

Desalting and buffer exchange of the Pfs25 of the activation reaction mixture can be accomplished by various membrane methods including dialysis and diafiltration with tangential flow membranes or by size exclusion chromatography. The activated Pfs25H is 0.2 micron filtered and is in 25 mM MES, pH 6.1 buffer. Its maleimide equivalents/Pfs25H (mol/mol) is in the range of 0.3 to 1.4. Again, it is understood that this is an average value reflecting all the Pfs25 forms in the activated Pfs25 sample. Lower or higher values may work equally well.

Activated Pfs25H could be separated from unactivated Pfs25H by RP-HPLC. The unactivated Pfs25H could be recycled. The site(s) of Pfs25H maleimide activation can be determined by peptide mapping.

Activation might also be carried out with immobilized Pfs25H, for instance, by binding the antigen through its polyHIS tag to a NiNTA resin. Such an activation may increase the reaction efficiency and aid in the desalting process.

As discussed above, Pfs25H has eleven disulfide bonds. The possibility of thiol-disulfide exchange was minimized by carrying out the maleimide-activated Pfs25H and thiolated iOMPC coupling at pH 6.1. The pH chosen for the reaction would also be influenced by reactants and product stability. In a control experiment no obvious evidence for thiol-disulfide exchange leading to stable mixed disulfide between unactivated Pfs25H and thiolated iOMPC was observed. Further, no differences could be detected in the properties of unactivated Pfs25 incubated with thiolated iOMPC under the same reaction conditions for the conjugation experiment when compared with the starting Pfs25 or Pfs25 incubated with control (not thiolated) iOMPC.

### Example 3

### Bromoacetyl-Activation of Pfs25H

Pfs25H was activated with bromoacetic acid N-hydroxysuccinimide ester at pH 7.3 with a 2.5 fold excess of reagent. Activated Pfs25H was desalted and was in 25 mM sodium borate, 0.15M NaCl, pH 8.0.

### Example 4

### Conjugation of Maleimide-Activated Pfs25H to Thiolated OMPC

All conjugation reactions were performed aseptically. In the first conjugation reaction (conjugate 1), the sEMCS-activated Pfs25H and the thiolated OMPC were mixed at 0.075 charge ratio of maleimide/thiol (mol/mol), and incubated overnight at 4° C. N-ethylmaleimide (NEM) was then added to quench the remaining thiols on the thiolated OMPC. The quenched solution was incubated overnight at 4° C., dialyzed against water then into 10 mM HEPES, 0.15M NaCl, pH7.3, and then spun at 1000×g for 5 minutes to remove any large aggregates. The Pfs25H-OMPC conjugate was in the supernatant.

In the second conjugation reaction (conjugate 2), the above conjugation reaction was repeated with the omission of the conjugate dialysis step into water and dialyzed directly into 10 mM HEPES, 0.15M NaCl, pH 7.3.

In the third conjugation reaction (conjugate 3), the quenching step is omitted. The sEMCS-activated Pfs25H and the thiolated OMPC were mixed at a 0.075 charge ratio of maleimide/thiol (mol/mol), and incubated for two days at 4° C. The mixture was then dialyzed first against Tris-HCl, sodium EDTA, sodium deoxycholate (TED) buffer and then against 10 nM HEPES, 0.15M NaCl, pH7.3, and then spun at 1000×g for 5 minutes. The Pfs25H-OMPC conjugate was in the supernatant.

In another conjugation reaction, the charge ratio of maleimide activated Pfs25H/thiolated OMPC was increased which afforded a conjugate with a higher Pfs25H loading (520 versus 325 Pfs25H/OMPC mol/mol). Animal studies using conjugates loaded with different amount of antigen can indicate a preferred amount of conjugate antigen loading to yield optimal immunogenic response.

### Example 5

### Conjugation of Bromoacetyl-Activated Pfs25H to Thiolated OMPC

Bromoacetyl-activated Pfs25H was mixed with thiolated OMPC in a 0.5 charge ratio by weight (activated Pfs25H/thiolated OMPC), and incubated for 2 days at 4° C. The mixture was then dialyzed first against TED buffer and then against 10 mM HEPES, 0.15M NaCl, pH 7.3, and then spun at 1000×g for 5 minutes. The Pfs25H-OMPC conjugate was in the supernatant.

### Example 6

### Analysis of the Pfs25H-OMPC Conjugates

6.1. Determining Aha/Pfs25H (mole/mole):

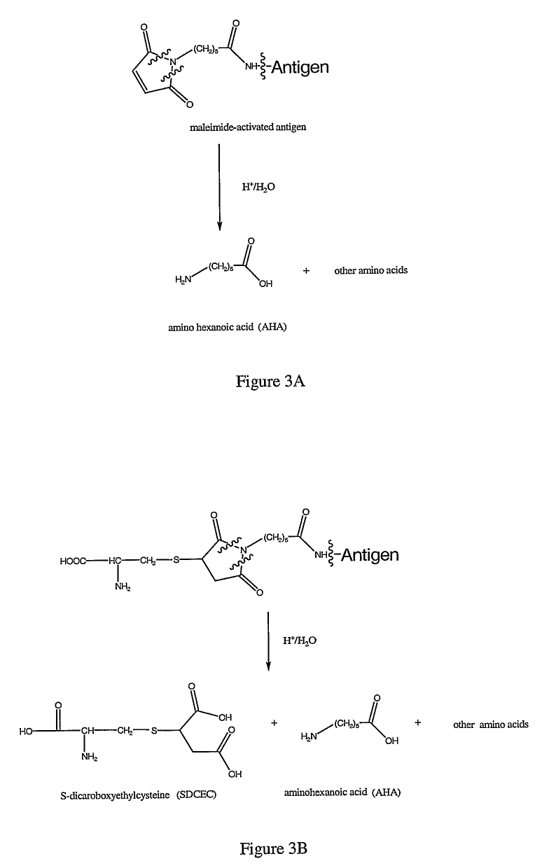

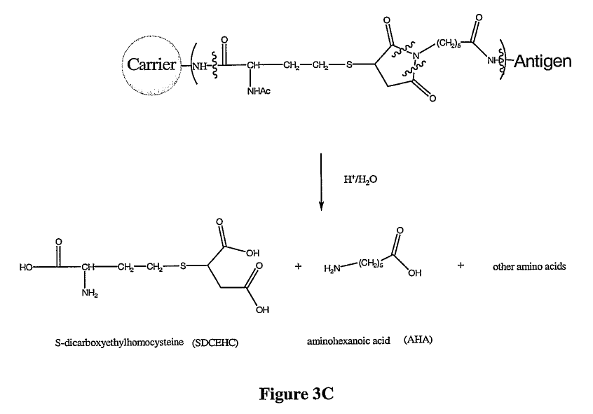

Aha is the product released from sEMCS maleimide-activated Pfs25H during acid hydrolysis. The concentration of Aha can be used to determine the extent of Pfs25H amine acylation by sEMCS. (FIG. 3A)

Maleimide-activated Pfs25H was acid hydrolyzed for 70 hours. From the hydrolytic products, the concentrations of experimental individual amino acids (nmol/mL; for eight amino acids: Gly, Ala, Val, Ile, Leu, Phe, Lys, and Arg) was obtained. The individual amino acid concentration is divided by the moles of that amino acid per mole of Pfs25H based on the Pfs25H sequence to get the concentration of Pfs25H For example, the Pfs25H concentration from the Leu data is: (367.70 nmol Leu/mL)/(9 mol Leu/mol Pfs25H)=29.8 nmol Pfs25H/mL. The average value obtained from the eight amino acids was: 29.5 nmol Pfs25H/mL.

The ratio of Aha to Pfs25H is then determined:

(19.5 nmol Aha/mL)/(29.5 nmol Pfs25H/ml)=0.66 Aha/Pfs25H

### 6.2. Determining the Reactive Maleimide Equivalents on Activated Pfs25.

In-process thiol consumption assay was used with N-acetylcysteine as the thiol and measuring remaining thiol using 5,5′dithiobis(2-nitrobenzoic acid) (DTNB). This can also be measured by amino acid analysis by reacting the Pfs25H maleimide with N-acetyl cysteine, desalting, acid hydrolysis, and measuring the thiol/maleimide reaction adduct hydrolysis product S-dicarboxyethyl cysteine (DCEC) see FIG. 3B. Similar assays can used for bromoacetamide activated malaria antigens with of course a change in the specific amino acid product analyzed.

MALDI-TOF MS and SDS-PAGE are useful tools to monitor reactivity of activated Pfs25H. Activated Pfs25H can be capped with an excess synthetic peptide containing no nucleophiles except a single cysteine. After the appropriate length of incubation excess peptide thiol is capped with excess NEM. The size of the peptide is chosen to provide appropriate mass resolution. Such an assay provides a facile method to optimize the malaria antigen activation reaction with respect to reagent concentration, reaction pH, and reaction time.

### 6. 3. Determining the Polypeptide Loading of Pfs25H/OMPC

Since maleimide activation of Pfs25 is heterogenous, no simple correlation of an incorporated amino acid, e.g. Aha and loading of antigen in the conjugate can be made. The multiple regression least squares analysis of amino acid analysis data for 70 h acid conjugate hydrolysates was performed to estimate Pfs25 levels in the conjugate (Shuler et al. J. Immunol Methods 156, 137-49 (1992)). A quantitative, competitive ELISA directed against the hexaHIS tag could prove useful in monitoring the conjugated Pfs25. For direct evidence for covalent bond formation between Pfs25H maleimide and thiolated OMPC measurement of the acid hydrolysis product S-dicarboxyethylhomocysteine (DCEHC), by Dionex Amino Acid Direct system can be performed (see FIG. 3C).

The weight ratio for a given Pfs25 (for example 239 mol/mol) loading can be calculated using 40×10⁶ g/mol OMPC and 20,446 g/mol Pfs25H:

(239 mol×20,446 g/mol Pfs25H)/(1 mol×40×10⁶ g/mol OMPC)=0.12

### 6. 4. Other Analytical Techniques

A modified Lowry protein assay was used to determine protein concentrations using BSA as a standard. In this modified assay, protein samples were precipitated with trichloroacetic acid in the presence of sodium deoxycholate (Bensadoun and Weinstein Anal. Biochem. 70, 241-250 (1976)). Protein pellets were dissolved in SDS-containing Lowry reagent A. SDS-PAGE was performed using pre-cast 4-20% Tris-glycine gels (Invitrogen). Samples were reduced using 0.1 M DTT and heated at 100° C. for 15 min and then electrophoresed using constant current (30 mA/gel). Gels were stained with Fast Stain (Zoion Research) according to manufacturer's instructions. Destained gels were digitized with a densitometer. For Western blots, gels were transferred to PVDF using a semi-dry apparatus (Invitrogen) and dry blocked and then incubated with appropriate murine primary antibodies in 5% non-fat dry milk with 0.04% (w/v)Tween 80. The blots were then washed with PBS and then incubated with goat anti-mouse HRP labeled secondary antibodies, washed with PBS, and then incubated with HRP substrate until bands were developed.

### Example 7

### Analysis of Bromoacetylated Pfs25H-OMPC Conjugates

The conjugate derived from the reaction of bromoacetyl-activated Pfs25H with thiolated OMPC did not yield quantifiable results. Amino acid analysis of the conjugate resulted in levels of S-carboxymethylhomocysteine (SCMHC) was below detection levels of the analysis system. SCMHC is the product released from the covalent coupling of bromoacetyl-activated antigens to thiolated OMPC (Marburg et al. J. Am. Chem. Soc. 108:5282-5287 (1986)).

Qualitative evidence for the conjugate exists, however. SDS-PAGE/FastStain and Western Blot results were consistent with the presence of conjugate. Overall, the bromoacetyl activated Pfs25H did not provide an efficient conjugation scheme as did the maleimide-activated Pfs25H.

### Example 8

### Properties of the Pfs25H-OMPC Conjugates

The conjugates from the first, second, and third were called conjugate 1, conjugate 2, and conjugate 3, respectively.

| | Conjugate 1 | Conjugate 2 | Conjugate 3 |
|---|---|---|---|
| Protein | 2.3 | 1.87 | 1.81 |
| Concentration | | | |
| (mg/ml) | | | |
| Pfs25H/OMPC | 239 ± 10 (n = 3) | 324 ± 46 (n = 4) | 324 (n = 1) |
| (mol/mol) | | | |
| Pfs25H/OMPC | 0.12 | 0.17 | 0.17 |
| (weight/weight) | | | |
| Residual Thiol | Not determined | <0.5% | <5% |
| (Residual/Starting) | | | |
| Size DLS Zaverage | 129 | 136 | 144 |
| (nm) | | | |
| Conjugate Yield | 40% | 44% | 42% |
| (mg conjugated Pfs/ | | | |
| mg starting Pfs) | | | |

The properties of the conjugates produced with maleimide/thiol coupling chemistry (conjugates 1, 2 and 3) were very similar. Through the analysis of SDS-PAGE/Fast Stain profiles, no difference between the conjugates was observed. Anti-NEM quenched OMPC mouse sera and anti-Pfs25H mAb 4B7 show similar reactivity with presumed conjugate bands for all three lots of Pfs25H/OMPC conjugates. Protective mouse sera from original vaccinations with conjugate 1 showed qualitatively similar reactivity for both conjugate 2 and conjugate 3 by Western blot.

In contrast, SDS-PAGE analysis suggested that lower reactivity between BrAc-Pfs25H and thiolated OMPC than for maleimide-activated Pfs25H.

### Example 9

### Formulations for the Pfs25H-OMPC Conjugate and Pfs25H

The conjugate was diluted with 1× saline to the concentration of 100 mcg/ml Pfs25H. The diluted conjugate solution was mixed at the ratio of 1:1 with 2×MAA in 1× Saline, and agitated gently for 2 hours at room temperature. The solution was then diluted with 1×MAA in 1× Saline, and agitated gently for 10 minutes at room temperature. The solution was then dispensed into vials.

| | Conjugate 1 | Conjugate 2 | Conjugate 3 |
|---|---|---|---|
| Alum Bound | >98% | 97% | 97% |
| Particle Size SLS | Not determined | 37 μm | 61 μm |
| vol. weighted mean | | | |

*Formulations had 48 mcg/mL Pfs25H; 283 mcg/mL OMPC

Pfs25H and A/Q OMPC were formulated separately to Pfs25H at 50 mcg/ml in 1×MAA, and A/Q OMPC at 372 mcg/ml in 1×MAA, and then mixed together at a ratio of 1:1 to get a blended formulation. The blended formulation was diluted with 1×MAA in 1× Saline to get the final formulation for unconjugated Pfs25H:OMPC vaccine.

Pfs25H protein was also aseptically homogenized at 8000 rpm at a designated final concentration in PBS and Montanide®ISA720 (SEPPIC, Paris, France) (30:70 aqueous to oil based on volume), or in PBS and Montanide®ISA51 (SEPPIC, Paris, France) (50:50 aqueous to oil based on volume), at room temperature for 3 minutes for 2 times, using a 50-mL stainless steel sealed chamber assembly attached to an Omni Mixer-ES homogenizer (Omni International, Warrenton, Va.). The mean emulsion droplet sizes were between 0.5 to 2.0 micrometer (μm), and by volume 90% of the droplets [D9v, 0.9)] are within 0.6 to 2.6 μm as measured using Mastersizer 2000 Particle Sizer Analyzer (Malvern Instruments Ltd. Malvern, UK).

### Example 10

### Animal Immunizations and Serum Collection

All animal studies were conducted in compliance with National Institutes of Health (NIH) guidelines (Guide for the Care and Use of Laboratory Animals, National Research Council, http://www.nap.edu/readingroomlbooks/labrats/; and Animal Welfare Act and Regulations, USDA, http://www.nal.usda.gov/awic/legislat/usdalegl.htm) and under the auspices of an Animal Care and Use Committee-approved protocols.

### 10. 1. Mouse Study.

Six- to 8 week-old female Balb/c mice were obtained from Taconic Farm (Germantown, Md.) and were maintained in the NIH animal facility during entire course of the studies. Mice were grouped randomly and were given one or two immunizations, either intraperitoneally or intramuscularly, of designated vaccines. Sera were collected from tail vein bleeds at the designated time points. Mice were bled prior to vaccination if a designated bleed time point falls on the same day of vaccination.

### 10. 2. Rabbit Study

Female New Zealand White rabbits of 2- to 3-kilograms were obtained from Covance (Denver, Pa.) and were maintained in the NIH animal facility during the entire course of the study. Rabbits were grouped randomly and were given intramuscularly 2 immunizations of designated vaccine. At designated time points auricular bleeds were taken from rabbits and the bleeds were processed for sera. Rabbits were bled prior to vaccination if a scheduled bleed time point falls on the same day of vaccination.

### 10. 3. Monkey Study.

Naïve Macaca mulatto monkeys were obtained from Morgan Island (S.C.) and were maintained in the NIH animal facility during the entire course of the study. Monkeys were grouped randomly and were given intramuscularly 1 or 2 immunizations of designated vaccines. Sera were obtained from bleeds taken from femoral region at designated time points. Monkeys were bled prior to vaccination if a designated bleed time point falls on the same day of vaccination.

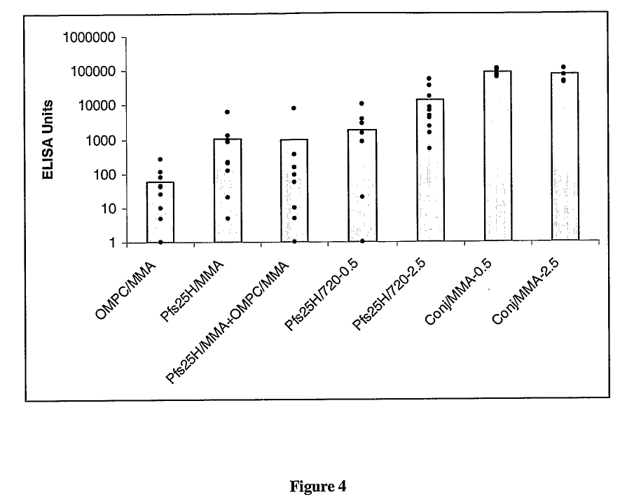

The conjugate was first tested in mice. Seven groups of mice, 10 in each group, were given by intraperitoneally 2 immunizations, on days 0 and 28, of test vaccine as indicated: OMPC/MAA (Merck Aluminum Adjuvant), 2.5 μg A/Q-OMPC adsorbed onto MAA; Pfs25H/MAA, 2.5 μg Pfs25H adsorbed onto MAA; Pfs25H/MAA+OMPC/MAA, 2.5 μg Pfs25 adsorbed onto MAA and 23 μg OMPC adsorbed onto MAA; Pfs25/720-0.5, 0.5 μg of Pfs25H formulated with Montanide®ISA720; Pfs25H/720-2.5, 2.5 μg of Pfs25H formulated with Montanide®ISA720; Pfs25H-OMPC Conj/MAA-0.5, 0.5 μg of OMPC-conjugated Pfs25H adsorbed onto MAA; and Pfs25H-OMPC Conj/MAA-2.5, 2.5 μg of OMPC-conjugated Pfs25H adsorbed onto MAA. Anti-Pfs25H antibody responses were evaluated in sera taken 2 weeks after the second immunization. The results were shown in FIG. 4.

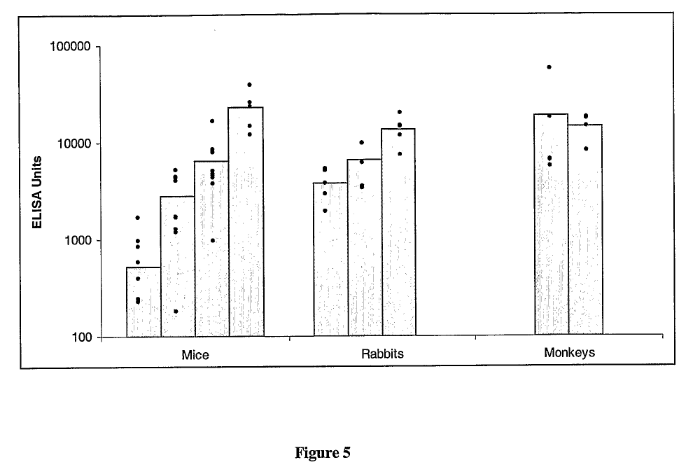

The immunogenicity and dose-response correlation of the conjugate in mice, rabbits, and rhesus monkeys was then tested and compared. Four groups of mice, 10 per group, were given intramuscularly (IM) 2 immunizations of 2, 10, 50, or 250 nanograms (ng) of conjugated Pfs25H (from left to right in FIG. 5) on study days 0 and 28. Anti-Pfs25H antibody responses were evaluated in sera taken 2 weeks after the second immunization. Three groups of rabbits, 5 per group, were given by IM 2 immunizations of 1, 5, or 20 micrograms of conjugated Pfs25H, from left to right, on study days 0 and 28. Anti-Pfs25H antibody responses were evaluated in sera taken 2 weeks after the second immunization. Five rhesus monkeys were given 4 μg and four were given 20 μg conjugated Pfs25H, by IM route, on days 0 and 70. Anti-Pfs25H antibody responses were evaluated in sera taken 2 weeks after the second immunization. The results were shown in FIG. 5. Numbers on the bar graph indicate the dose (in microgram) of conjugated Pfs25H given to the animal group.

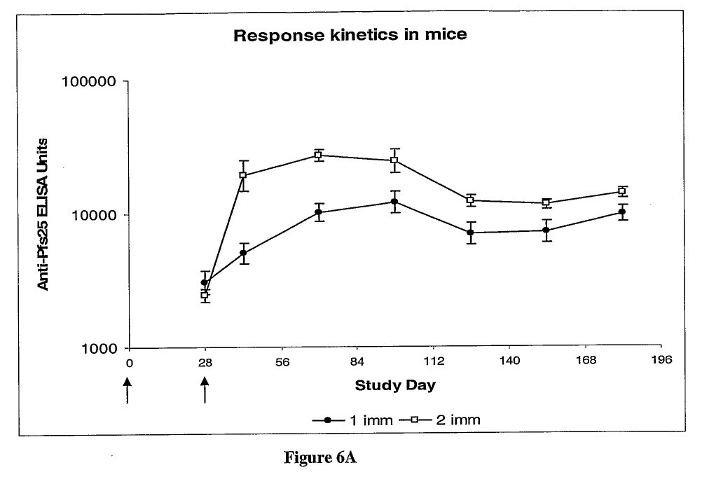

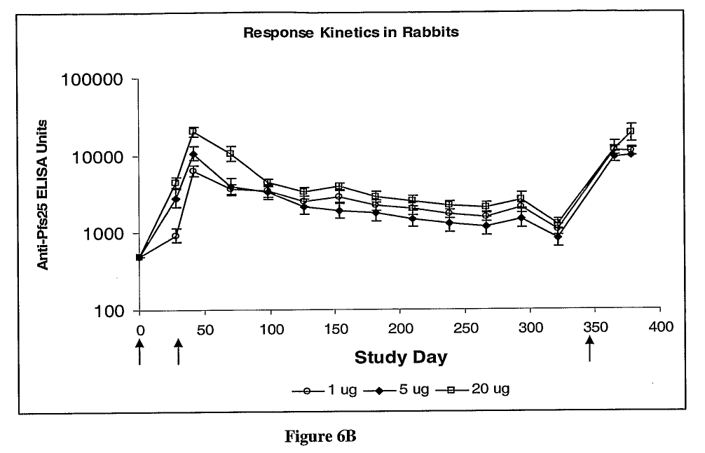

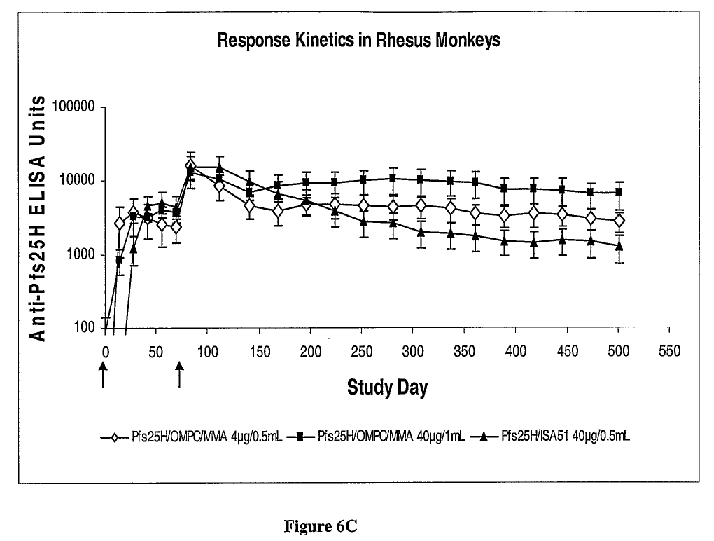

Antibody responses time course for the conjugate was analyzed also in mice, rabbits, and rhesus monkeys, respectively. Two groups of mice were given one or two immunizations of 0.25 μg of conjugated Pfs25H. The second immunization was given 4 weeks after the first immunization. Three groups of rabbits were given 2 immunizations of 1, 5, or 20 μg of conjugated Pfs25H on study days 0 and 28. To investigate whether the antibody responses can be boosted by the vaccine, one additional dose of 1 μg conjugated Pfs25 was given to all groups of rabbits on study day 350. For the rhesus study, the animals were given 2 immunizations of 4 or 20 μg conjugated Pfs25H on study days 0 and 70. Sera collected at the indicated time points were analyzed for anti-Pfs25H levels. The results were shown in FIG. 6A-6C. Arrows indicate the day of immunizations.

### Example 11

### Enzyme-Linked Immunosorbent Assay (ELISA)

Antibody levels were measured in serum by a standardized ELISA. ELISA plates (Immunolon 4; Dynex Technology Inc., Chantilly, Va.) were coated with Pfs25H or OMPC, stored at 4° C. overnight, then blocked with buffer containing 5% skim milk (Difco, Detroit, Mich.) in Tris-buffered saline (TBS) (BioFluids, Camarillo, Calif.) for 2 hours at room temperature. Sera diluted in buffer, were added in triplicate to antigen-coated wells and incubated for 2 hours at room temperature. After washing with 0.1% Tween 20 in TBS, plates were incubated with alkaline phosphatase-labeled goat anti-human IgG (Kirkegaard & Perry, Gaithersburg, Md.) for 2 hours at room temperature. After adding phosphatase substrate solution (Sigma, St. Louis, Mo.), absorbance was read at 405 nm.

The ELISA was standardized using reference antisera with assigned unit value equivalent to the reciprocal of the dilution giving an OD₄₀₅ of 1. In each ELISA plate, the absorbances of a set of serially diluted reference were fitted to a four parameter hyperbolic function to generate a standard curve. Using this standard curve, the absorbance of an individual test serum was converted to an antibody unit value.

### Example 12

### Transmission Blocking Assay (TBA)

The transmission blocking activity of the animal sera was tested by an ex vivo membrane feeding assay.

The test sera from animal studies were heat-inactivated and diluted with a Type O naïve human serum pool in a ratio as indicated. The diluted test serum was mixed with an in vitro gametocyte culture of P. falciparum (NF54 isolate), and the mixture was fed to Anopheles stephensi mosquitoes through a membrane feeding apparatus. Mosquitoes were kept for 8 to 10 days after the feed to allow parasite to develop into mature oocysts. Infectivity was measured by dissecting at least 20 mosquitoes per serum sample, staining the midgut with Mercurochrome, and counting the number of oocysts per midgut. The negative control feed uses Type O human sera from individuals who have never been exposed to malaria. Percent inhibition of oocyst development per mosquito was determined by the formula: 100×(Mean oocyst No. in negative control sample−Mean oocyst No in test sample)/Mean oocyst No. in negative control sample

### Example 13

### Pfs25H-OMPC Conjugate Induced Potent Antibody Responses in Mice, Rabbits and Rhesus Monkeys

The immunogenicity of Pfs25H-OMPC conjugate was compared with that of Pfs25H in the water-in-oil emulsion Montanide®ISA720 (ISA720) formulation, which was the best performer in previous studies. The route of injection (intraperitoneal, IP), and the starting doses of 0.5 or 2.5 μg of conjugated Pfs25H were selected as these are routine potency assay parameters for the ISA720 formulation.

As shown in FIG. 4, the antibody responses induced by 0.5 μg conjugated Pfs25H were about 10 times higher than those induced by 2.5 μg of Pfs25H/ISA720. Since the responses seemed to be saturated at the 0.5 μg conjugated Pfs25 dose, the enhancement magnitude is likely to be higher than 50 times. As a negative control, mice were immunized with un-conjugated OMPC adsorbed onto Merck Aluminum Adjuvant (MAA). Production of anti-OMPC antibodies were confirmed by ELISA using OMPC as coating antigen. The anti-OMPC antibodies did not cross-react with Pfs25H antigen. Physical mixing of Pfs25H and OMPC adsorbed onto MAA only generated antibody responses comparable to those induced by Pfs25H/MAA, indicating chemical conjugation is required for the immune enhancement.

The Pfs25H-OMPC conjugate elicited specific antibodies in mice and rabbits in a dose-dependent fashion, whereas the dose range given to Rhesus monkeys reached in plateau in antibody responses (FIG. 5). The conjugate induced antibody levels peaked at 2 to 4 weeks after the second immunization and persisted at least 5 to 6 months after the immunizations (FIGS. 6A and 6B). Giving Rhesus monkeys one immunization with the conjugate induced a lasting antibody response up to at least 70 days. A second dose given on the study day 70 resulted in a remarkable boost of antibody responses (FIG. 6C). Interestingly, the antibody level in mice given one dose of the Pfs25H-OMPC conjugate was so persistent that at the end of study (day 182), it was comparable to those induced by 2 immunizations.

### Example 14

### Antibodies Induced by Conjugates are Potent Transmission Blockers

The correlation between anti-Pfs25 ELISA units and the percent inhibition of oocyst density in mosquitoes, i.e. transmission blocking activity, fits a hyperbolic dose-response curve [K. Miura, and A. Saul, unpublished data] Approximately 3000 (for anti-Pfs25 sera from rabbits immunized with Pfs25/ISA7200 or 6000 (for anti-Pvs25 sera from monkeys immunized with Pvs25/CpG) antibody units give 80% inhibition of oocyst density per mosquito. As shown in Table 1, the anti-sera induced by Pfs25-OMPC conjugate were highly potent transmission blocker.

TABLE 1
Transmission blocking activity of mouse sera
immunized with Pfs25H-OMPC conjugate.

| | | Anti-Pfs25 | | |
|---|---|---|---|---|
| | | ELISA | Oocyst | % |
| Test sera | Dilution^{a} | Unit | Number^{b} | Inhibition^{c} |
| Type O+ naïve human | N/A | | 5.3 | N/A |
| serum pool | | | | |
| Anti-OMPC sera^{d} | 1:12 | | 7.30 | 0 |
| | 1:24 | | 5.25 | 0 |
| | 1:48 | | 9.68 | 0 |
| | 1:96 | | 10.35 | 0 |
| | 1:192 | | 7.00 | 0 |
| Anti-Pfs25H- | 1:12 | 2991 | 0.00 | 100.00 |
| OMPC conjugate | 1:24 | 1495 | 0.03 | 99.52 |
| sera^{e} | 1:48 | 748 | 0.08 | 99.23 |
| | 1:96 | 374 | 0.50 | 95.17 |
| | 1:192 | 187 | 1.28 | 81.79 |

^{a}Dilutions were made by mixing with Type O+ naïve human serum pool. The ratio indicates the volume of test serum:diluent.
^{b}Average of oocyst number in >20 mosquitoes fed with the test serum.
^{c}% Inhibition = 100 × (Mean oocyst No. in negative control samples − Mean oocyst No in test sample)/ Mean oocyst No. in negative control sample.
^{d}Serum pool from mice immunized with OMPC adsorbed onto MAA.
^{e}Serum pool from mice immunized with 2.5 μg of conjugated Pfs25H.

Three lots of conjugates from different conjugation processes induced comparable immune responses, indicating that conjugation processes are robust.

Other embodiments are within the following claims. While several embodiments have been shown and described, various modifications may be made without departing from the spirit and scope of the present invention.

1. A Malaria antigen-carrier conjugate comprising
a carrier protein, and
a plurality of Plasmodium antigen polypeptides, wherein each of the antigen polypeptides is a wild-type antigen protein expressed in the sexual stage of Plasmodium or a derivative of the wild-type antigen protein, wherein each of the antigen polypeptides may be the same, or different; and
wherein the plurality of Plasmodium antigen polypeptides are covalently linked to the carrier protein.

2. The conjugate of claim 1 wherein the Plasmodium is selected from the group consisting of P. falciparum and P. vivax.

3. The conjugate of claim 2 wherein the plurality of Plasmodium antigen polypeptides comprise a derivative from an antigen selected from the group consisting of Pfs25, Pfs28, Pfs48/45, Pvs25 and Pvs28.

4. The conjugate of claim 3 wherein the plurality of Plasmodium antigen polypeptides comprise a Pfs25H protein produced in P. pastoris or S. cerevisiae.

5. The conjugate of claim 4 wherein the plurality of Plasmodium antigen polypeptides consist essentially of a Pfs25H protein produced in Pichia pastoris.

6.-8. (canceled)

9. The conjugate of claim 4 wherein the plurality of Plasmodium antigen polypeptides comprise a Pvs25H protein produced in S. cerevisiae.

10. The conjugate of claim 1 wherein the plurality of Plasmodium antigen polypeptides comprise derivatives of Pfs25 and Pvs25.

11. The conjugate of claim 1 wherein the plurality of Plasmodium antigen polypeptides consist essentially of derivatives of Pfs25 and Pvs25.

12. The conjugate of claim 1 wherein the carrier protein is selected from the group consisting of OMPC (Outer Membrane Protein Complex of Neisseria meningitidis), iOMPC (improved OMPC), BSA (bovine serum albumin), OVA (ovalbumin), THY (bovine thyroglobulin), KLH (keyhole limpet hemocyanin), TT (tetanus toxoid protein), HBsAg (surface antigen protein) and HBcAg (core antigen protein) of Hepatitis B virus, rotavirus capsid proteins, the L1 protein of the human papilloma virus, and VLP (virus-like particle) type 6, 11 and 16.

13.-14. (canceled)

15. The conjugate of claim 1 wherein the plurality of Plasmodium antigen polypeptides are covalently linked to the carrier protein via a thioether linker.

16. A malaria antigen-carrier conjugate which is produced by a method comprising conjugating to a carrier protein a plurality of Plasmodium antigen polypeptides, wherein each of the antigen polypeptides is a wild-type antigen protein expressed in the mosquito stage of Plasmodium or a derivative the wild-type antigen protein, wherein each of the antigen polypeptides may be the same, or different.

17. The conjugate of claim 16 wherein the plurality of Plasmodium antigen polypeptides comprise Pfs25H.

18. The conjugate of claim 16 wherein the carrier protein is OMPC.

19. The conjugate of claim 16 wherein the conjugating is achieved through a scheme selected from the group consisting of maleimide/thiol coupling, bromoacetamide/thiol coupling, and histidine-selective cross-linking.

20. The conjugate of claim 16 wherein the conjugating is achieved using a cross-linker selected from the group consisting of one of more of sulfosuccinimidyl 4-(N-maleimidomethyl)cyclohexane-1-carboxylate (sSMCC), N-maleimidobenzoyl-N-hydroxysuccinimide ester (MBS), glutaraldehyde, 1-ethyl-3-(3-dimethylaminopropyl)carbodiimide (EDCI), Bis-diazobenzidine (BDB), N-acetyl homocysteine thiolactone (NAHT), and N-[ε-Maleimidocaproyloxy]sulfosuccinimide ester (sEMCS).

21. The conjugate of claim 20 wherein the cross-linker is N-[ε-Maleimidocaproyloxy]sulfosuccinimide ester (sEMCS).

22. A vaccine against malaria comprising a conjugate according to claim 1, an adjuvant and a physiologically acceptable carrier.

23. The vaccine of claim 21 the conjugate is a Pfs25H-OMPC conjugate.

24. The vaccine of claim 21 wherein the conjugate is absorbed onto Merck aluminum adjuvant.

25. The vaccine of claim 22 further comprises a Pvs25-OMPC conjugate.

25.-27. (canceled)
